(19) United States
(12) Patent Application Publication
(10) Pub. No.: US 2004/0095615 A1
(43) Pub. Date: May 20, 2004

(54) Biological scanner with two dimensional scanning operation

(76) Inventor: Chung-Hua Tsai, Hsinchu City (TW)

Correspondence Address: SUPREME PATENT SERVICES, POST OFFICE BOX 2339, SARATOGA, CA, 95070, US

(21) Appl. No.: 10/295,084

(22) Filed: Nov. 14, 2002

(51) Int. Cl. G06K 9/00; C12M 1/34

(52) U.S. Cl. 358/474; 435/287.1

(57) ABSTRACT

A scanner includes a casing defining an interior space in which an optic module movable in a longitudinal direction by means of a first transmission system is mounted. A carrier is arranged inside the casing below the optic module and is selectively movable out of the casing by means of a second transmission system. A tray supports an array of cells arranged in rows and columns for each receiving and retaining a biological sample to be scanned. The tray is movably mounted on the carrier and movable with respect to the carrier in a transverse direction by a third transmission system. The optic module is movable with respect to the sample cells in a line-by-line manner in the longitudinal direction, the tray being movable with respect to the optic module in a line-by-line manner in the transverse direction for covering a two-dimensional area in which the article is located.

### FIELD OF THE INVENTION

The present invention relates generally to a scanner for scanning biological samples arranged in two dimensional array, and in particular to a scanner capable of two dimensional scanning operation for obtaining images of the biological samples deposited deep in deposition cells.

### BACKGROUND OF THE INVENTION

Optical scanners that are currently available in the market comprise an optic module movable in a longitudinal direction. The optic module comprises a line array of light emitting elements that extend in a transverse direction in a span covering a width of a document to be scanned. Thus, by means of the longitudinal movement of the optic module, the document is scanned in a line-by-line manner. However, it is noted that the optic module of the conventional optical scanner comprises a single lens for focusing light reflected from the document to an optic detector, such as charge-coupled device (CCD). This makes it impossible to obtain non-distorted image of a sample deposited deeply inside a deposition cell, which is often encountered in scanning biological samples.

Further, for documents that contain deeply raised/recessed patterns, the conventional scanners that have a single lens cannot obtain image without distorting the deeply raised/recessed patterns.

Thus, it is desired to have a scanning device for overcoming the above problems.

### SUMMARY OF THE INVENTION

An object of the present invention is to provide a scanner capable of obtaining non-distorted images of samples arranged in a two dimensional array.

Another object of the present invention is to provide a scanner capable of obtaining a non-distorted image of a deeply raised/recessed pattern formed on a plain document.

A further object of the present invention is to provide a biological scanner that is internally mounted in a personal computer.

To achieve the above objects, in accordance with the present invention, there is provided a scanner comprising a casing defining an interior space in which an optic module movable in a longitudinal direction by means of a first transmission system is mounted. A carrier is arranged inside the casing below the optic module and is selectively movable out of the casing by means of a second transmission system. A tray supports an array of cells arranged in rows and columns for each receiving and retaining a biological sample to be scanned. The tray is movably mounted on the carrier and movable with respect to the carrier in a transverse direction by a third transmission system. The optic module is movable with respect to the sample cells in a line-by-line manner in the longitudinal direction, the tray being movable with respect to the optic module in a line-by-line manner in the transverse direction for covering a two-dimensional area in which the article is located.

### BRIEF DESCRIPTION OF THE DRAWINGS

The present invention will be apparent to those skilled in the art by reading the following description of a preferred embodiment thereof, with reference to the attached drawings, in which:

FIG. 1 is a perspective view of a scanner constructed in accordance with the present invention;

FIG. 2 is an exploded view of the scanner of the present invention;

FIG. 3 is a perspective view of the scanner illustrating the operation of moving a sample tray in and out of a casing of the scanner;

FIG. 4 is a perspective view of the scanner illustrating the operation of transverse movement of the sample tray;

FIG. 5 is a perspective view of the scanner illustrating longitudinal movement of an optic module of the scanner;

FIG. 6 is a schematic side elevational view illustrating scanning operation of the scanner over deep cells in which biological samples are deposited; and

FIG. 7 is a perspective view of a computer in which the scanner of the present invention is mounted.

### DETAILED DESCRIPTION OF THE INVENTION

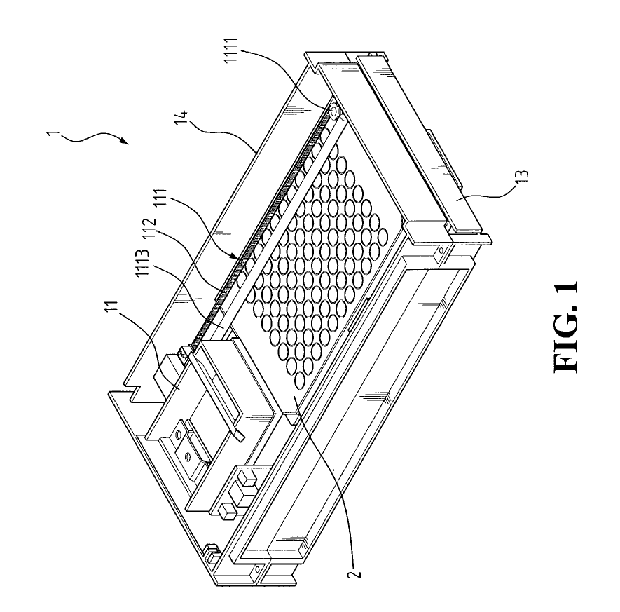

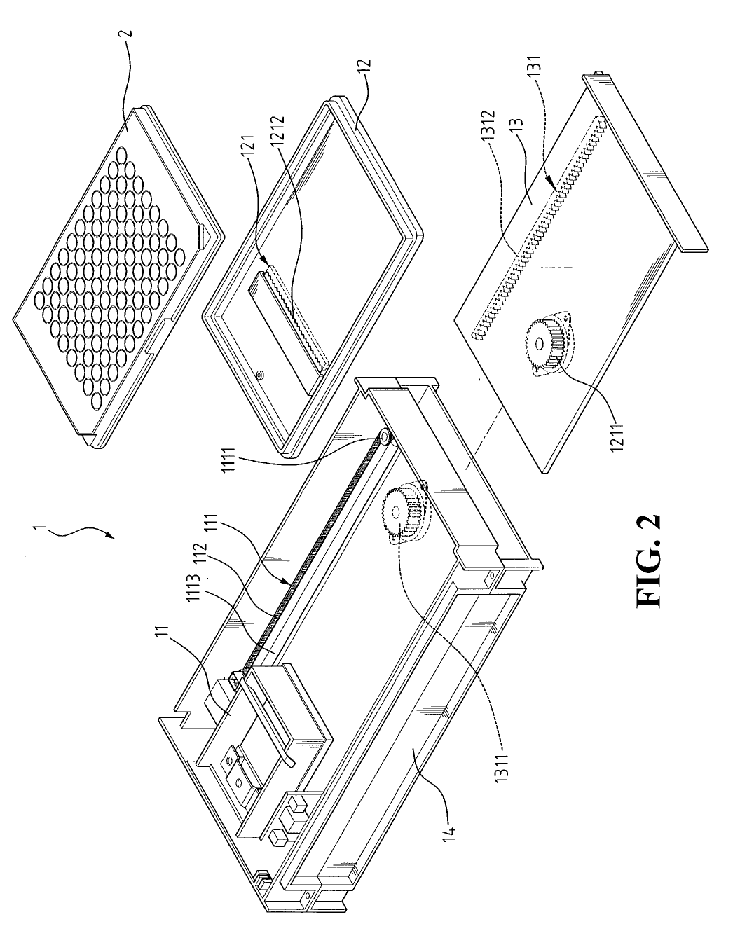

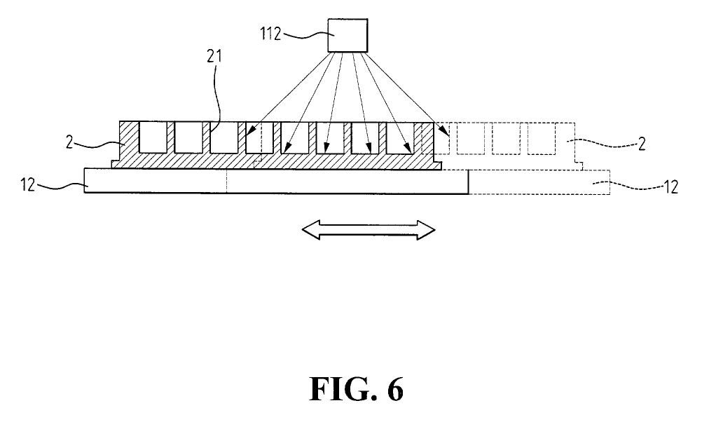

With reference to the drawings and in particular to FIGS. 1 and 2, a scanner constructed in accordance with the present invention, generally designated with reference numeral 1, comprises a casing 14 defining an interior space (not labeled) in which an optic module 11 is mounted. The optic module 11 is of the conventional type and construction, comprising a light source 112 (FIG. 6) for selectively projecting a light beam toward a sample to be scanned and a lens for focusing the light reflected from the sample to an optic detector, such as charge-coupled device, for formation of an image of the scanned sample. Since the construction of the optic module 11 constitutes no novel part of the invention, no further detail will be given hereinafter.

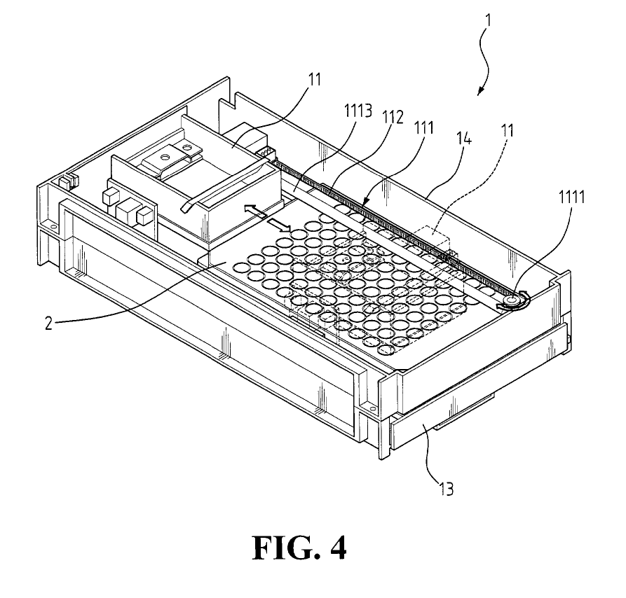

The scanner 1 comprises a longitudinal transmission system 111 for moving the optic module 11 in a longitudinal direction with respect to the casing 14, as indicated by double arrows of FIG. 4. The longitudinal transmission system 111 comprises a timing belt 1112 and associated toothed wheels 1111 around which the timing belt 1112 surrounds. Rails 1113 are mounted in the casing 14 for supporting and guiding the longitudinal movement of the optic module 11. The longitudinal transmission system 111 is coupled to the optic module 11 for driving the optic module 11 along the rails 1113. It is apparent to those having ordinary skills to replace the toothed wheels 1111 with friction wheels for frictionally driving a belt that takes the place of the timing belt 1112.

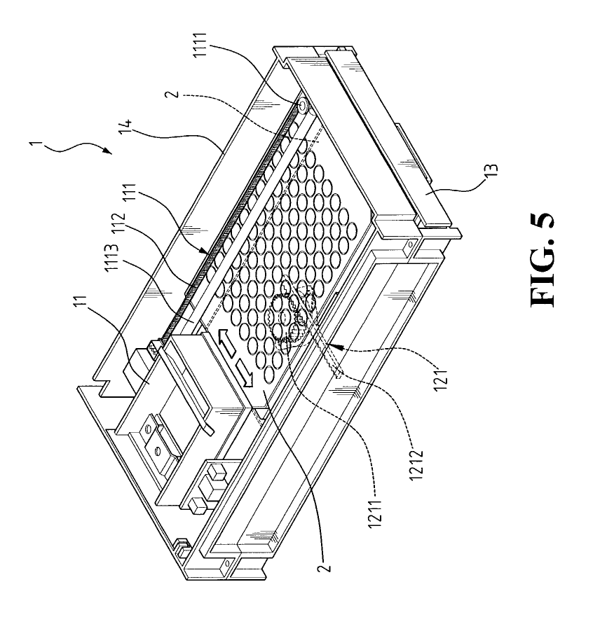

A sample tray 12 is arranged below the optic module 11. A sample holder 2 defining a plurality of cells 21 (FIG. 6) is positioned on and supported by the sample tray 12. Each cell 21 receives and retains a sample to be scanned. The scanner 1 comprises a transverse transmission system 121 for moving the sample tray 12 that is located below the optic module 11 in a transverse direction with respect to the casing 14, as illustrated by double arrows shown in FIG. 5. The transverse transmission system 121 comprises a rack 1212 mounted on an underside of the sample tray 12 and extending in a transverse direction. A gear 1211 is rotatably mounted on a carrier 13 (to be further described) and is engageable with the rack 1212 whereby when the gear 1211 is rotated, the sample tray 12 is transversely moved with respect to the casing 14.

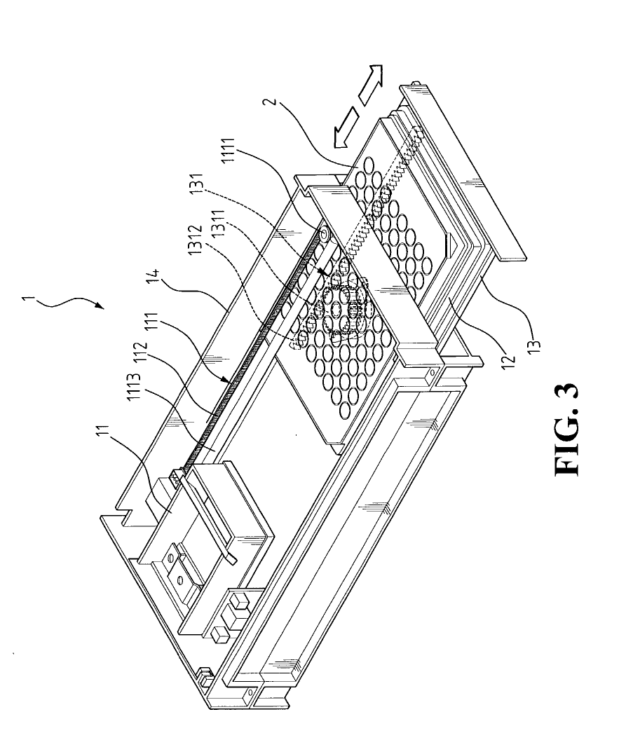

The carrier 13 is movably arranged inside the casing 14 for moving into and out of the casing 14 as indicated by double arrows of FIG. 3. The sample tray 12 is positioned on the carrier 13 and is movable therewith. Thus, the transverse movement of the carrier 13 drives the sample tray 12 and the sample holder 2 in and out of the casing 14. The scanner 1 further comprises an additional transmission system 131 comprising a rack 1312 extending in the longitudinal direction and mounted on an underside of the carrier 13. A gear 1311 is mounted on a bottom of the casing 14 and is engageable with the rack 1312. The engagement between the rack 1312 and the gear 1311 drives the sample tray 12 in and out of the casing 14.

Also referring to FIG. 3, to operate, the carrier 13 (together with the sample tray 12 positioned thereon) is moved out of the casing 14 first for loading the sample holder 2 in which samples to be scanned are deposited in the cells 21. The carrier 13 is moved back into the casing 14 again.

Also referring to FIG. 4, the optic module 11 is moved in the longitudinal direction to go over the sample cells 21 in a row-by-row manner along a given column that extends in the longitudinal direction. Once the scanning operation on each cell of the column is completed, the optic module 11 is back to its initial position and the sample tray 12 moved in the transverse direction to have the next column of the cell array aligned with the optic module 11 for scanning the cells of the column as shown in FIG. 5. In this way, the rows and columns of the cell array can be completely scanned in a row-by-row and column-by-column manner.

Also referring to FIG. 6, when the optic module 11 is positioned at any row, it is not possible for the lens 112 of the optic module 11 to obtain a complete image of each cell 21 of the row due to the depth of the cells 21. Thus, the transverse movement of the sample tray 12 as indicated by arrow of FIG. 6 helps aligning each cell 21 of the row with respect to the lens 112 one by one.

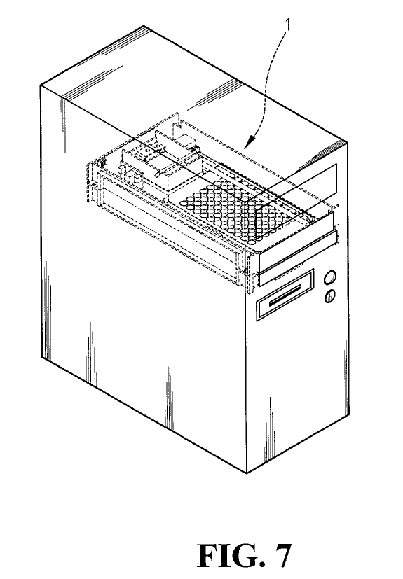

FIG. 7 shows an application of the scanner 1 of the present invention. The scanner 1 is made as an internal peripheral device for a personal computer and can be secured in a disk cage of the computer. Thus, biological samples can be deposited in the cells 21 of the sample holder 2 and loaded into the computer for performing scanning operation over the samples under the control of the computer. No wire connection between the scanner 1 and the computer is needed. In addition, the scanner 1 can be readily removed from the personal computer for maintenance and repairing.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A scanner comprising:
a casing defining an interior space;
an optic module movable in a longitudinal direction by means of a first transmission system;
a carrier selectively movable out of the casing by means of a second transmission system;
a tray adapted to support at least one article to be scanned, the tray being movably mounted on the carrier and movable with respect to the carrier in a transverse direction by a third transmission system;
wherein the optic module is movable with respect to the at least one article in a line by line manner in the longitudinal direction, the tray being movable with respect to the optic module in a line by line manner in the transverse direction for covering a two-dimensional area in which the article is located.

2. The scanner as claimed in claim 1, wherein the first transmission system comprises toothed wheels and a timing belt engaging the wheels, a rail being mounted inside the casing for guiding the longitudinal movement of the optic module.

3. The scanner as claimed in claim 1, wherein the second transmission system comprises a rack extending in the longitudinal direction and mounted on an underside of the carrier and a gear mounted on a bottom of the casing and engaging the rack.

4. The scanner as claimed in claim 1, wherein the third transmission system comprise a rack extending in the transverse direction and mounted on an underside of the tray and a gear mounted on the carrier and engaging the rack.

5. The scanner as claimed in claim 1, further comprising a sample holder positioned on the tray, the sample holder defining an array of cells having rows and columns, each cell adapted to receive and retain a sample.

6. The scanner as claimed in claim 1, wherein the sample is a biological sample.
